(19) United States
(12) Patent Application Publication
(10) Pub. No.: US 2009/0036470 A1
(43) Pub. Date: Feb. 5, 2009

(54) PALIPERIDONE DERIVATIVES

(76) Inventors: Jiri Bartl, Strelice (CZ); Frantisek Picha, Brno (CZ)

Correspondence Address: SYNTHON IP INC, 7130 HERITAGE VILLAGE PLAZA, STE 202, GAINESVILLE, VA, 20155, US

(21) Appl. No.: 12/179,695

(22) Filed: Jul. 25, 2008

Related U.S. Application Data
(60) Provisional application No. 60/952,376, filed on Jul. 27, 2007.

(51) Int. Cl. A61K 31/519; C07D 239/70; A61P 25/00

(52) U.S. Cl. 514/259.41; 544/282

(57) ABSTRACT

A compound of formula (1) is useful as a pharmaceutical and as an intermediate in making paliperidone.

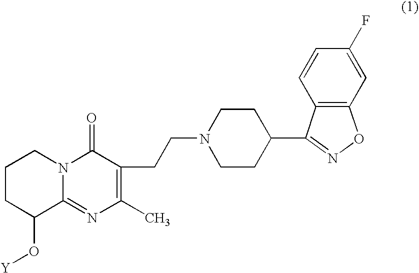

wherein Y is an acid-sensitive group of the formula:

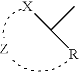

in which X represents an oxygen atom, a sulfur atom, or an —NH— group; Z represents a C2-C7 alkylene bridge; and R represents hydrogen or a C1-C4 alkyl group.

This application claims the benefit of priority from prior U.S. Provisional Application Ser. No. 60/952,376, filed Jul. 27, 2007, the entire contents of which being incorporated herein by reference.

Paliperidone, or 9-hydroxyrisperidone (chemically: (±)-3-(2-(4-(6-fluoro-1,2-benzisoxazol-3-yl)-1-piperidinyl)ethyl)-6,7,8,9-tetrahydro-9-hydroxy-2-methyl-4H-pyrido[1,2-a]pyrimidin-4-one) of the formula (A):

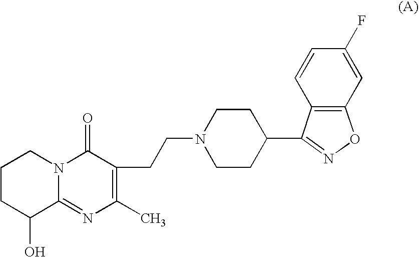

is a major metabolite of the known antipsychotic drug risperidone. It has also been developed as an active pharmaceutical ingredient and is currently marketed in an extended-release tablet under the brand name INVEGA® by Ortho-McNeil-Janssen Pharmaceuticals, Inc.

Paliperidone has one center of optical activity (the C—OH carbon in the 9-position of the pyrido[1,2-a]pyrimidin-4-one ring moiety). The single enantiomers are known but the marketed compound is a racemate.

Paliperidone (including enantiomeric forms thereof) has been disclosed in EP 368388 (U.S. Pat. No. 5,158,952). The same document also discloses esters of paliperidone with carboxylic acids having the formula (B) (R⁴═C₁₋₁₉ alkyl) (both racemic esters and single enantiomers).

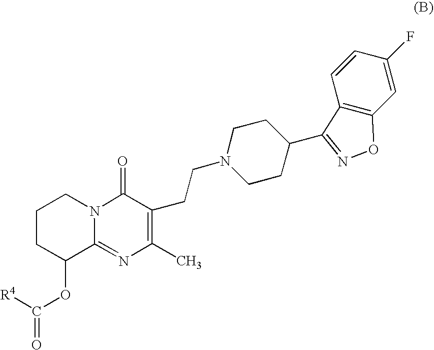

A preferred ester compound is paliperidone palmitate, particularly for injectable compositions.

WO 95/14691, EP 730594, U.S. Pat. No. 5,688,799 relate to novel O-alkylated derivatives of the formula (C) (and hydrogenated analogues thereof)

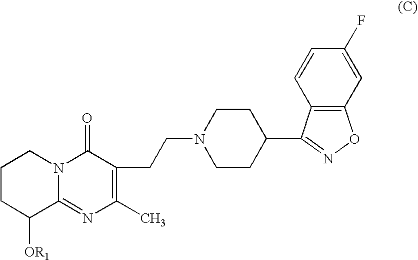

The compounds (C) have an ether substituent on the 9-position of the pyrido[1,2-a]pyrimidin-4-one ring moiety. R₁ represents C2-C6 alkenyl; C2-C6 alkynyl; C3-6 cycloalkyl optionally substituted with C1-4 alkyl; or C1-19 alkyl optionally substituted with C3-C6 cycloalkyl, halo, C1-C6 alkyloxy or cyano. Among others, 9-methoxy and 9-propoxy analogues are preferred.

### SUMMARY OF THE INVENTION

The present invention relates to compounds that are useful as antipsychotic agents, as prodrugs for paliperidone, and/or as intermediates in making paliperidone. A first aspect of the invention relates to a compound of formula (1) or a salt thereof:

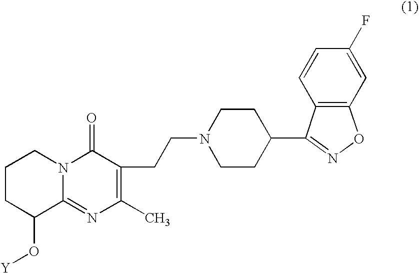

wherein Y is an acid sensitive group of the formula:

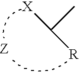

in which X is oxygen, sulfur or —NH— group, Z represents a C2 to C7 alkylene bridge, and R represents hydrogen or C1-C4 alkyl group. The alkylene bridge can be branched to form a carbon chain (generally C1-C2) substituent on the C—X-Z ring. Typically X is oxygen, Z is unbranched and R is hydrogen. One preferred compound is represented by formula (2), wherein X is oxygen, Z is 4 carbons, and R is hydrogen:

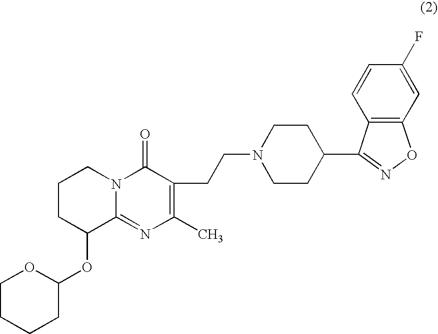

The compounds of formula (1) and their pharmaceutically acceptable salts can be used as a pharmaceutical agent in a pharmaceutical composition; e.g., in conjunction with at least one pharmaceutically acceptable excipient. Pharmaceutical compositions comprising the compound (1) as the active ingredient form a specific aspect of the present invention.

Another aspect of the invention relates to various processes for making the compounds of formula (1). These processes include:

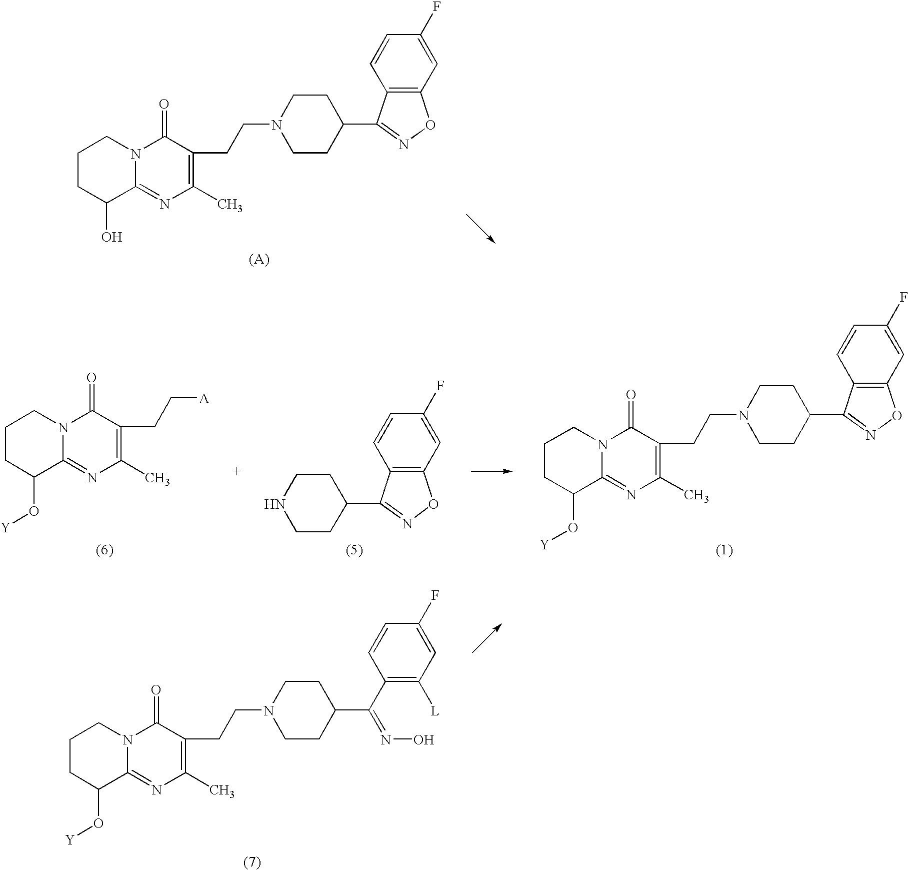

wherein the leaving groups “A” and “L” are defined below.

In a third aspect, the invention deals with a process of converting the compound of formula (1) into paliperidone. The process comprises subjecting a compound of formula (1) to acidic hydrolysis to form a compound of formula (A):

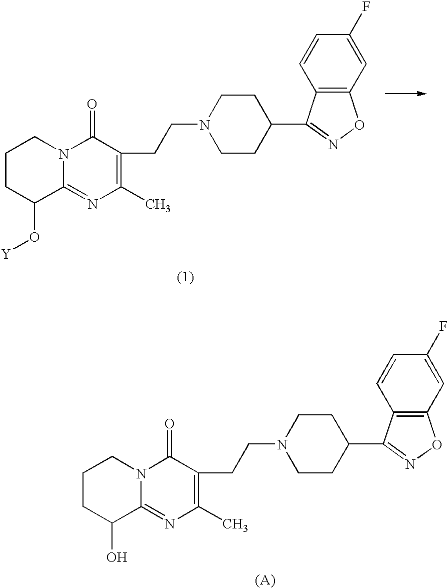

The cleavage of the group Y (a replacement of the group Y by hydrogen) from the compound (1) by an acidic hydrolysis can be performed in a variety of environments and generally by a strong acid. In a specific aspect, this process is performed by the stomach environment after a peroral administration of the compound (1) to a human or animal patient.

### BRIEF DESCRIPTION OF THE DRAWINGS

FIG. 1 shows the conversion of the compound (2) into paliperidone from the orally disintegratable formulation of Example 5.

FIG. 2 shows the overall release of paliperidone from the formulation of Example 5.

### DETAILED DESCRIPTION OF THE INVENTION

As used throughout this application and the claims, the structural formulas other than formula (3) further include the salts thereof unless expressly limited or the context indicates otherwise, such as in the examples. The occasional reference to formula compounds and their salts is intended as a reminder of the inclusion of salts and no adverse or exclusive meaning is intended when such language is omitted in mentioning a formula compound. Rather, for convenience, the otherwise ubiquitous refrain of “and salts thereof” has been frequently omitted in order to improve readability.

The present invention involves compounds of the general formula (1)

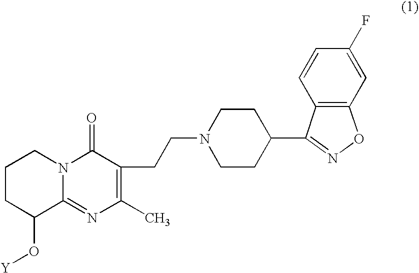

the salts thereof, especially pharmaceutically acceptable salts thereof, and the individual as well as mixed stereochemically isomeric forms thereof. The compounds of formula (1) are characterized by having an acid-sensitive group Y:

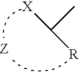

in which X represents an oxygen atom, a sulfur atom, or an —NH— group; Z represents a C2-C7 alkylene bridge; and R represents hydrogen or a C 1-C4 alkyl group.

The group Y is “acid sensitive” in that the whole group Y may be split off and replaced with hydrogen, to generate an —OH group, by treatment with an acid, particularly with a strong acid, such as hydrochloric acid, sulfuric acid and the like. The reaction can take place in a solid and/or liquid phase. By such cleavage, paliperidone of formula (A) is formed as shown below.

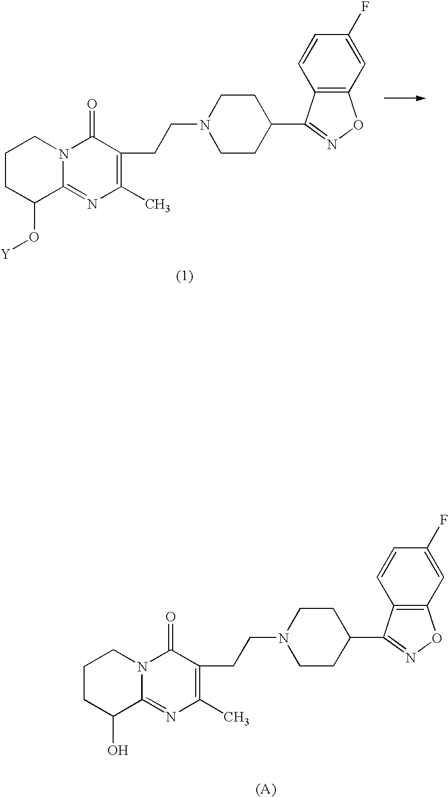

Advantageously, the compounds of the formula (1) are so designed that the group Y may be split under conditions that correspond to the conditions of the stomach environment. These characteristics may simply be tested by an in vitro test, wherein the compound (1) is subjected to a reaction with 0.1 N HCl or with another simulated gastric fluid, at the temperature of 37° C. and at the concentration of the compound (1) that corresponds to a conventional use of the drug for the therapeutic purposes (2 to 50 mg per 1 liter of the acid fluid). Advantageously, the Y group is sufficiently acid sensitive that it is cleaved, and paliperidone liberated, in at least 80% conversion, within 2 hours or less, more preferably within 30 minutes or less.

Typically in the Y group, X is oxygen and R is hydrogen. The alkylene bridge, Z, is usually unbranched but can have one or more branches which result in monovalent alkyl substituent(s) on the ring. The total number of carbons, however, including the branched chains, in the Z moiety is within the range of 2-7. Typically Z is an unbranched, C3-C5 alkylene chain. A particularly preferred compound wherein Z is C4 is a compound of formula (2)

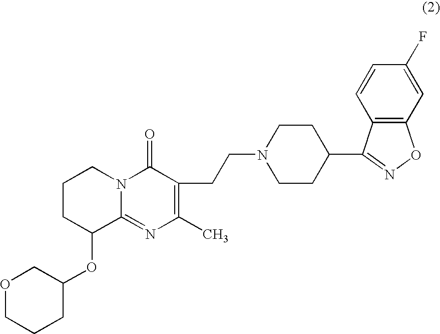

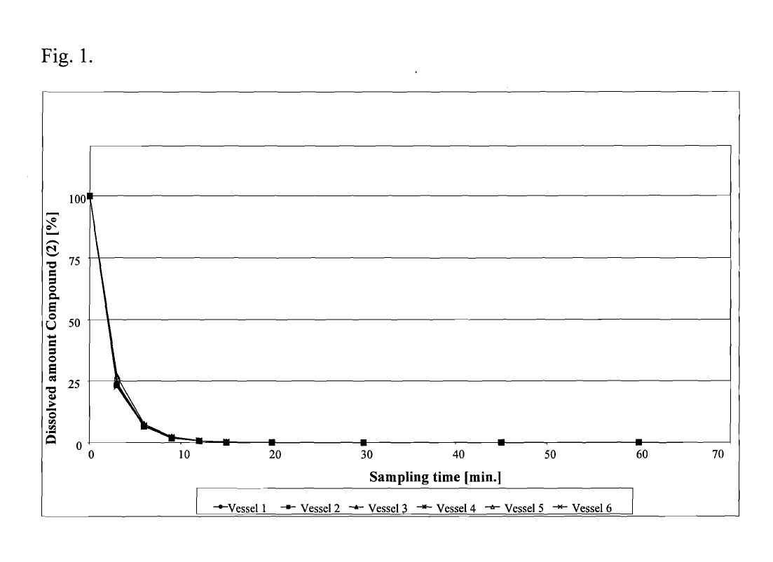

Thus Y is preferably tetrahydropyranyl. The compound (2) can be converted to paliperidone quantitatively such as shown in FIG. 1. The compounds of formula (1) thus may serve as oral prodrugs of paliperidone.

The YO— moiety, although acid sensitive, may be less reactive under a variety of circumstances than the corresponding HO— group in paliperidone. In particular, the compounds of the invention may be less sensitive to aerial oxygen and moisture and less sensitive to interaction with certain excipients, e.g., with basic excipients, in pharmaceutical formulations. Therefore, the compounds of the invention may offer improved stability vis-a-vis paliperidone in certain respects.

The compounds of formula (1) and some of their intermediates discussed in detail later have two stereogenic centers (carbon 9 and the asymmetric carbon in the group Y); thus they may exist as diastereomers. The absolute configuration on this center may be indicated by stereochemical descriptors R and S according to the known rules.

Pure stereochemically isomeric forms of said compounds and said intermediates can generally be obtained by the application of art-known procedures. For example, diastereoisomers can be separated by physical methods such as selective crystallization or chromatographic techniques, e.g., counter current distribution, liquid chromatography and the like methods.

Pure stereochemically isomeric forms of the compounds of formula (1) may also be obtained from the pure stereochemically forms of the appropriate intermediates and starting materials, provided that the intervening reactions occur stereospecifically.

The pure and mixed stereochemically isomeric forms of the compounds of formula (1) are intended to be embraced within the scope of the present invention.

The invention also embraces various solid state forms of the compounds of formula (1) including salts, solvates and hydrates thereof.

The compounds of the general formula (1) may be produced by various processes. In the following preparations, the reaction products may be isolated from the reaction medium and, if necessary, further purified according to methodologies generally known in the art such as, for example, extraction, crystallization, trituration and chromatography. The compounds may be isolated as solids, either as crystalline or as amorphous materials. A solid form of the compounds, if obtainable, is the preferred form. Due to the intended industrial application, compounds with more than 95% chemical purity, and particularly with more than 99% chemical purity are preferred.

In a first process of making compounds of the formula (1), paliperidone of formula (A) or a salt thereof reacts with the donor of the Y group, hereinafter a “Y-donor,” to form a compound of formula (1) or a salt thereof. Generally two kinds of Y-donor compounds are readily used in the process. The first Y-donor has the formula Y-LV, wherein Y is the acid sensitive group as defined above and LV is a suitable leaving group, e.g., a halogen group such as fluoro, bromo, or chloro, or an alkyl- or arylsulfonyloxy (hereinafter simply “sulfonyloxy”) group such as methanesulfonyloxy, trifluoromethane-sulfonyloxy, benzenesulfonyloxy, or 4-methylbenzenesulfonyloxy. The reaction can be represented as follows:

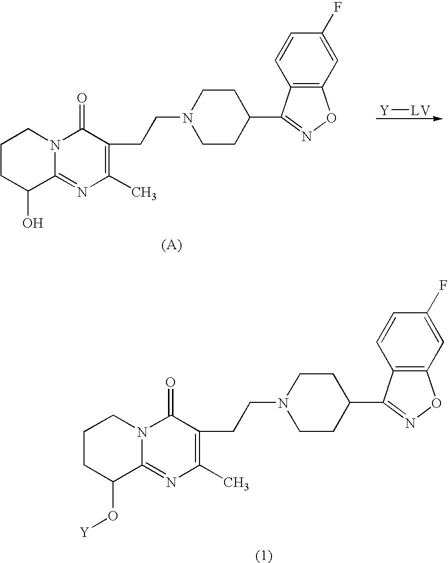

A second Y-donor, suitable where R is hydrogen, is a compound of formula (Y1):

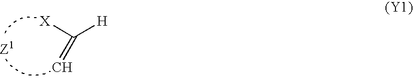

wherein Z¹ represents a C2-C6 alkylene bridge and X represents oxygen, sulfur or —NH— group. In a specific example, (Y1) represents a dihydropyran compound of the formula (3).

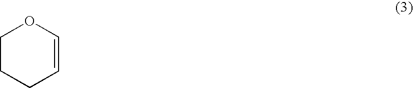

The use of a compound of formula (3) as the Y-donor in a reaction with paliperidone (A) yields the tetrahydropyranyl-substituted paliperidone of the formula (2), as shown below.

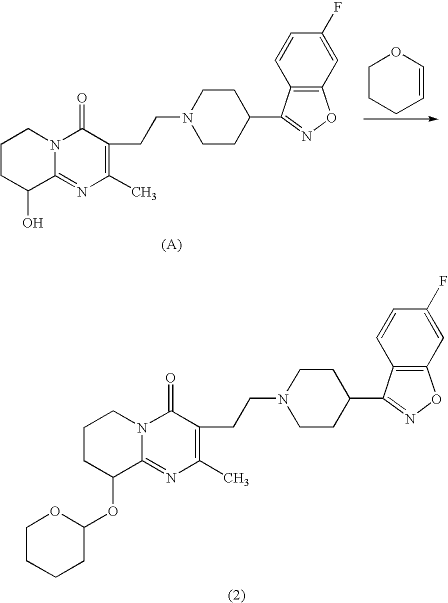

The process of making the compound (1) typically comprises contacting, under reactive conditions, the paliperidone compound (A) or an acid addition salt thereof, e.g., paliperidone hydrochloride, and the Y-donor, typically the dihydropyran compound (3), in an inert solvent, e.g., in an C1-C10 aliphatic or aromatic hydrocarbon or halogenated hydrocarbon (e.g., benzene, toluene, hexane, heptane, petroleum ether, chloroform, chlorobenzene, dichloromethane). In some embodiments the reaction is carried out under catalysis with a strong acid (trifluoroacetic acid, benzenesulfonic acid, etc.).

The starting paliperidone may be prepared by the processes known in the art, preferably by an alkylation of a 3-piperidinyl 1,2-benzisoxazole of the formula (4) with the compound of formula (5), wherein A represents an appropriate leaving group such as, for example, a halogen, e.g., chloro, bromo or iodo; sulfonyloxy group, e.g., methanesulfonyloxy, trifluoromethane-sulfonyloxy, benzenesulfonyloxy, 4-methylbenzenesulfonyloxy; or the like leaving groups.

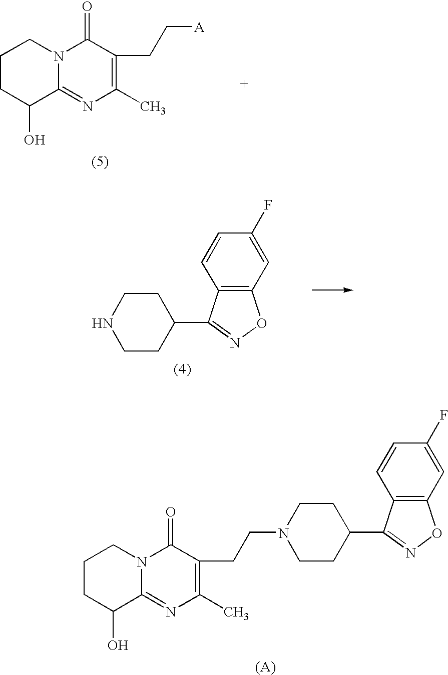

In a second process for making the compounds of formula (1), the starting material is a novel compound of formula (6), which may be prepared by the reaction of the compound of formula (5), in which A is as defined above, with a Y-donor as defined above. In a typical embodiment, the reaction proceeds as follows:

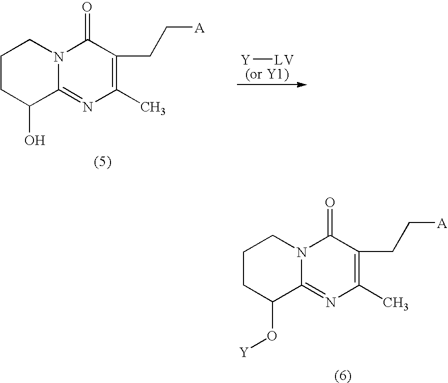

The substituents A- and Y- in the compounds of formula (6) are identical with those as defined above.

The compound (5) has been disclosed, e.g., in EP 368388. The formula (5) also comprises acid addition salts, e.g., a hydrochloride. A typical compound of the formula (5) is one where A is chlorine and is represented by the formula (5a)

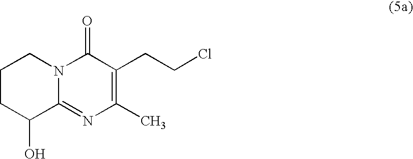

The formulas (5) and (6) comprise at least one stereogenic centre and therefore they include also the single enantiomers and mixtures thereof.

In a specific example, the Y-donor is advantageously a dihydropyran compound, typically of the formula (3). Accordingly, one preferred embodiment of the compound of formula (6) is a compound of formula (6a) or a salt thereof.

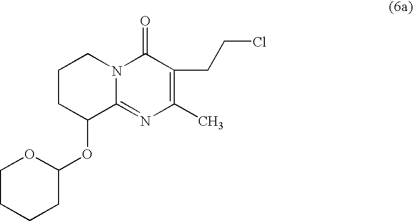

The reaction of the compound (5) with the Y-donor generally proceeds in an inert reaction solvent, e.g., in a hydrocarbon (such as in benzene, toluene, petroleum ether, hexane, heptane, etc.), or halogenated hydrocarbon (chloroform, dichloromethane, etc.). If the Y-donor is the compound of the formula (Y1), such as the compound of formula (3), the reaction is advantageously catalyzed by an acid.

The compound of formula (6), once prepared, N-alkylates the compound of formula (4), yielding the desired compound of formula (1) as illustrated below.

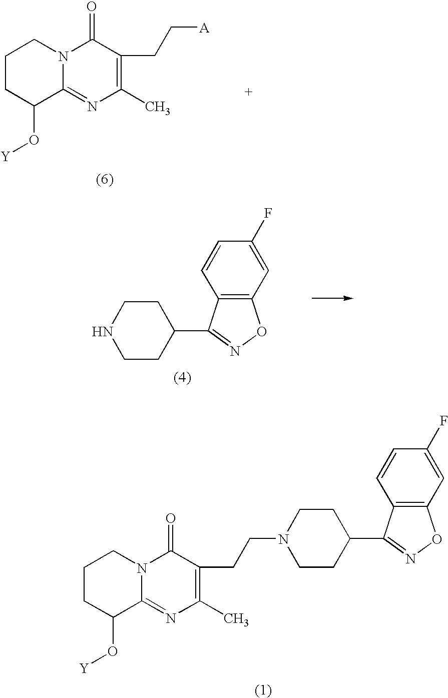

The N-alkylation reaction can conveniently be carried out by mixing the reactants in a reaction-inert solvent such as, for example, water; an aromatic solvent, e.g., benzene, toluene, xylene, chlorobenzene and the like; a C1-6 alkanol, e.g., methanol, ethanol, 1-butanol and the like; a ketone, e.g., acetone, 4-methyl-2-pentanone and the like; an ester, e.g., ethyl acetate and the like; an ether, e.g., diethylether, tetrahydrofuran, 1,4-dioxane and the like; a dipolar aprotic solvent, e.g., N,N-dimethylformamide, N,N-dimethylacetamide, dimethylsulfoxide and the like; a tertiary amine, e.g., pyridine and the like; a nitrile, e.g., acetonitrile and the like; or a mixture of such solvents. The addition of an appropriate base such as, for example, an alkali metal or an earth alkaline metal carbonate, hydrogen carbonate, hydroxide, oxide, carboxylate, alkoxide, hydride or amide, e.g., sodium carbonate, sodium hydrogen carbonate, potassium carbonate, sodium hydroxide, calcium oxide, sodium acetate, sodium methoxide, sodium hydride, sodium amide and the like, or an organic base such as, for example, a tertiary amine, e.g., triethylamine, pyridine and the like, may optionally be used to trap the acid which is formed during the course of the reaction. Stirring and elevated temperatures (up to the reflux temperature of the reaction mixture) may enhance the rate of the reaction. The reaction may be catalyzed by an inorganic iodide, e.g., by potassium iodide. Additionally, it may be advantageous to conduct said N-alkylation under an inert atmosphere such as, for example, oxygen-free argon or nitrogen gas. Alternatively, the N-alkylation may be carried out by applying the conditions of phase transfer catalysis reactions. These conditions comprise stirring the reactants, with an appropriate base and optionally under an inert atmosphere as defined hereinabove, in the presence of a suitable phase transfer catalyst such as, for example, a trialkylphenyl-methylammonium, tetraalkylammonium, tetraalkylphosphonium, tetraarylphosphonium halide, hydroxide, hydrogen sulfate and the like catalysts.

In a specific example of the second process, the compound of formula (6a) reacts with the compound of formula (4), yielding the compound of formula (2).

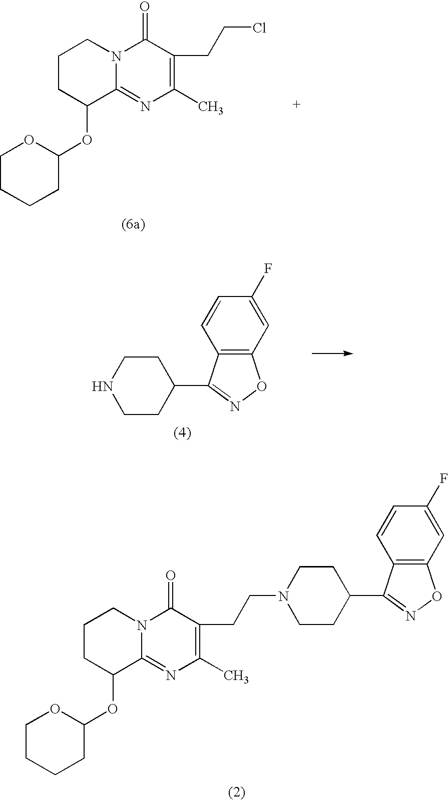

In a third process, the compounds of formula (1) can also be obtained by the cyclization of an oxime of formula (7), wherein L is a reactive leaving group such as, for example, halogen or nitro group.

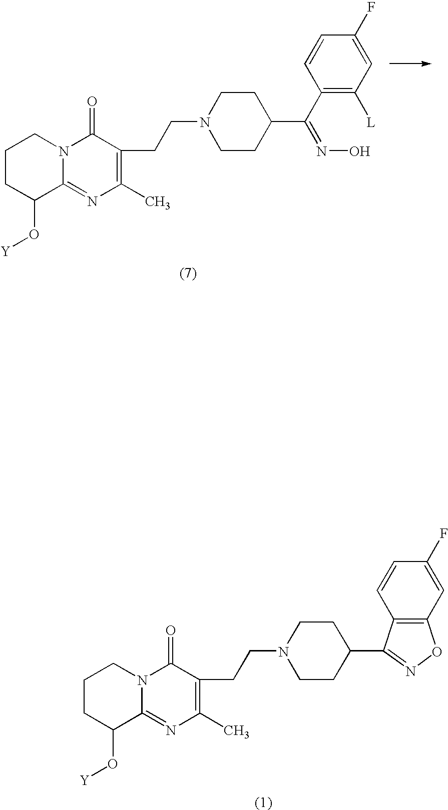

Preferably L is a halogen group and more particularly a fluoro group. The formula (7) contains two stereogenic carbon centers and therefore it also includes single diastereomers and mixtures thereof. Furthermore, the formula (7) comprises also a C═N—OH bond that may exist in an E- or Z-configuration. The preferred configuration is the Z-configuration, which is typically more reactive in the cyclization reaction.

The formula (7) also comprises acid addition salts and any solid state forms, hydrates and solvates.

The cyclization reaction may conveniently be conducted by treatment with an appropriate base, preferably in a suitable reaction-inert solvent at temperatures in the range of 20° C. to 150° C., and in particular at the reflux temperature of the reaction mixture. Advantageously, the base may first be added, preferably at room temperature, whereupon the thus formed oxime salt is cyclized, preferably at an increased temperature and more preferably at the reflux temperature of the reaction mixture.

Appropriate bases for the cyclization are, for example, alkali and earth alkaline metal carbonates, hydrogen carbonates, hydroxides, alkoxides or hydrides, e.g., sodium carbonate, sodium hydrogen carbonate, potassium carbonate, sodium hydroxide, sodium methoxide, sodium hydride or organic bases such as amines, e.g., triethylamine, pyridine and the like bases. Suitable solvents are, for example, water; aromatic hydrocarbons, e.g., benzene, toluene and the like; halogenated hydrocarbons, e.g., dichloromethane, chloroform, 1,2-dichloroethane and the like; 1-6 carbon alkanols, e.g., methanol, ethanol, and the like; ketones, e.g., acetone, 4-methyl-2-pentanone and the like; ethers, e.g., 1,4-dioxane, tetrahydrofuran and the like; dipolar aprotic solvents, e.g., N,N-dimethyl-formamide, N,N-dimethylacetamide, dimethylsulfoxide, 1-methyl-2-pyrrolidinone and the like, or mixtures of such solvents.

The compounds of general formula (7) are novel compounds. They may be prepared by an N-alkylation reaction of the oxime compound of formula (8) with the compound of formula (6) defined above.

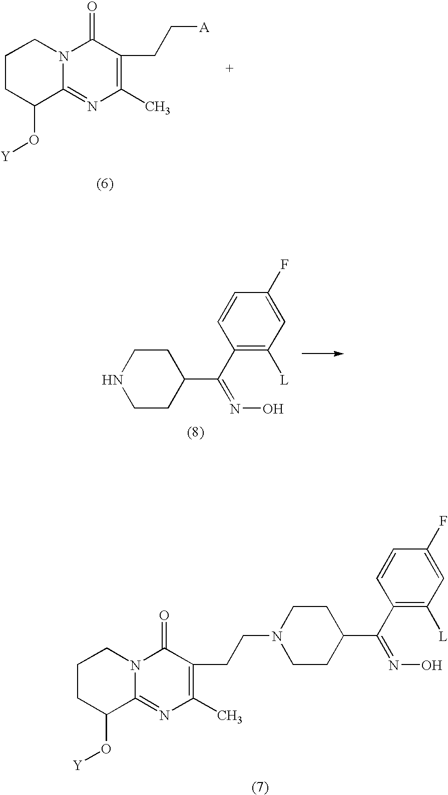

The compounds of general formula (8) are known in the art, e.g., from EP 196132. A preferred example is the compound of formula (8a), more preferably the (Z)-isomer thereof.

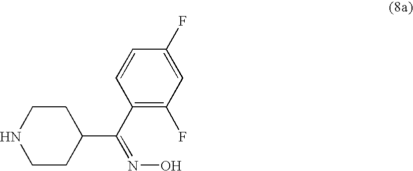

The conditions of the N-alkylation reaction are essentially the same as disclosed above for the N-alkylation reaction of the compound (6) with the compound (4).

The preferred compound of the general formula (7) is the compound of formula (7a), which results from the reaction of the oxime compound (8a) with the compound (6a)

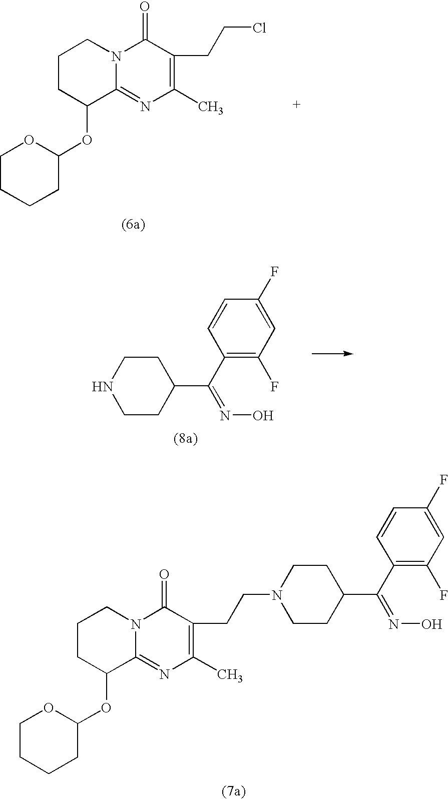

Under the general conditions disclosed above, the compound (7a), and preferably the (Z)-isomer thereof, cyclizes to yield the compound of the formula (2)

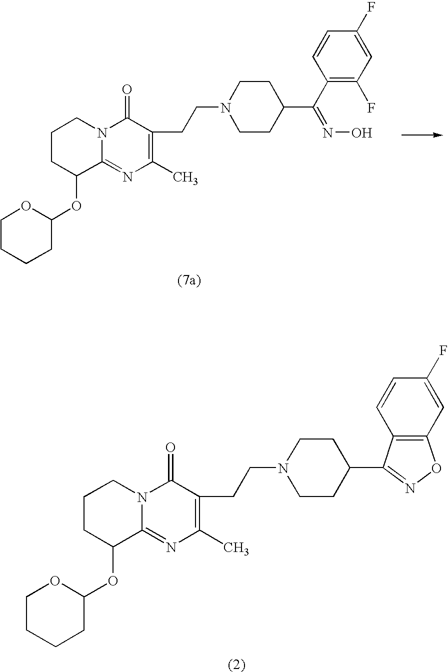

As pointed out above, the compounds of formula (1) exhibit an acid sensitive group Y and therefore they may be converted into paliperidone in vivo, i.e., in the acidic environment of the stomach. Alternatively, they may also serve as intermediates in making paliperidone in vitro, particularly in an industrial production process.

In such a process, the compounds of the general formula (1), and particularly the preferred compound of the formula (2), may be converted to paliperidone by treatment with an acid in a suitable solvent. Suitable acids are, e.g., hydrochloric, hydrobromic, sulfuric, phosphoric acid, organic carboxylic acid such as formic, acetic, trifluoroacetic acid, organic sulfonic acids such as methane sulfonic, benzene sulfonic, toluene sulfonic acid, and the like.

The hydrolytic cleavage reaction is preferably conducted in a suitable solvent such as water, an alcohol, an ester, a ketone, a nitrile, a hydrocarbon, and so on. Preferred solvents are water, C1-C6 aliphatic alcohol and mixtures thereof.

Advantageously, the reaction with the acid proceeds at ambient and/or slightly enhanced (up to 50° C.) temperature.

In particular, the acid chosen for the cleavage may be an optically active acid, such as tartaric acid, etc. Herein, the acidic cleavage into the paliperidone may be accompanied with a resolution of paliperidone into enantiomers, as the resulting racemate of paliperidone may form a diastereomeric pair of salts with the optically active acid, wherein one member of the diastereomeric pair of salts may preferentially precipitate from the reaction mixture.

The paliperidone, however obtained and whether in enantiomerically pure form or not, may be isolated from the reaction mixture and purified, if desired, by known processes.

The compounds of formula (1) have pharmacological activity as an antipsychotic and/or as a prodrug for the antipsychotic paliperidone and thus may also be formulated into various pharmaceutical compositions and forms. In particular, these compositions and forms are usually designated for peroral administration, but the invention is not limited thereto. As pointed out above, the compounds of the present invention may serve as prodrugs for paliperidone, which may be converted into paliperidone by the stomach acid. In addition, they may be converted into paliperidone by an enzymatic cleavage, thus it is not excluded that the compounds of the invention may be also administered directly into the systemic blood circulation, e.g., by injections or by subcutaneous application.

The pharmaceutical compositions of the invention comprise a compound of formula (1) or a pharmaceutically acceptable acid addition salt thereof and at least one pharmaceutically acceptable excipient. For example, in preparing the compositions in an oral dosage form, any of the usual solid carriers, binders, release control agents, disintegrating agents and the like may be employed. Because of their ease in administration, tablets and capsules represent the most advantageous oral dosage unit form. The tablets and capsules may be designated for immediate release (disintegrating either in mouth or in stomach), or for prolonged release.

For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed.

Compositions for subcutaneous administration comprise various injectable depot forms, from which the active substance is slowly released into the systemic blood circulation.

In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in a dosage unit form for ease of administration and uniformity of dosage. Dosage unit form as used herein refers to physically discrete units suitable as unitary dosages, each unit containing a predetermined quantity of active ingredient calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof. A dosage unit contains an effective amount of the compound of formula (1) or a salt thereof, typically in the range of 0.1 mg to 100 mg, in combination with at least one pharmaceutically acceptable excipient as explained above.

In view of the usefulness of the subject compounds in the treatment of psychotic diseases, it is evident that the present invention provides a method of treating warm-blooded animals suffering from psychotic diseases, said method comprising administration of an antipsychotic amount of a compound of formula (1) or a pharmaceutically acceptable acid addition salt thereof, effective in treating diseases associated with the release of neurotransmitters, in particular psychotic diseases. Those of skill in the treatment of such diseases can determine a suitable and/or effective amount. In general, it is contemplated that an effective antipsychotic amount would be from about 0.01 mg/kg to about 4 mg/kg body weight, more preferably from about 0.04 mg/kg to about 2 mg/kg body weight.

The invention will be described with respect to the following non-limiting examples.

### EXAMPLES

### Example 1

Preparation of Compound (2) from Paliperidone

Reaction Scheme:

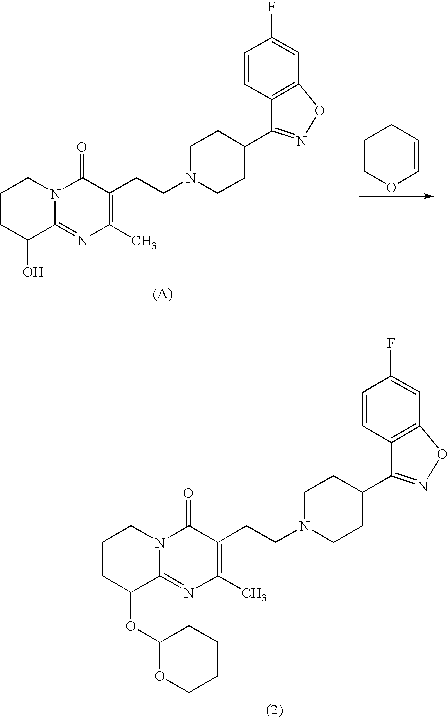

Procedure:

1.00 g of paliperidone hydrochloride was suspended in 10 ml of chloroform. To this, 0.3 ml of 3,4-dihydro-2H-pyran and 0.29 g of benzenesulfonic acid were added at 20° C. Reaction mixture was agitated overnight. 0.38 ml of triethylamine was added, then the solution was extracted with 25 ml of 3% sodium bicarbonate. Water layer was extracted with 2*10 ml of chloroform. Chloroform solution was extracted with 2*10 ml of water and dried with sodium sulfate. The solution was evaporated at 70° C.

Crude product was purified by a column chromatography (Silica gel 60 (0.040-0.063 mm), mobile phase EtOAc/MeOH/Et₃N 150/5/2 v/v) under TLC control (mobile phase EtOAc/MeOH/Et₃N 10/3/0.5 v/v).

### Example 2

Preparation of Compound (6a) from Compound (5a)

Reaction Scheme:

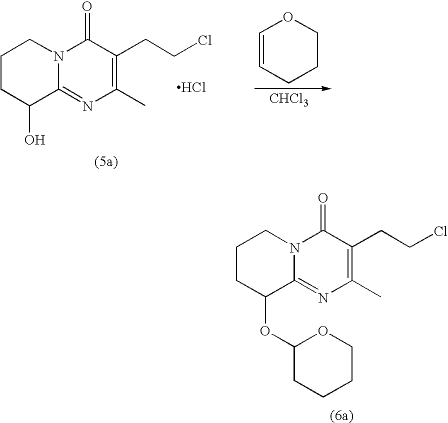

Procedure:

30.00 g of (5a) was dissolved in 100 ml of chloroform and, after dissolution, 20 ml of dihydropyran and 1.70 g of benzenesulfonic acid were added. Exothermic reaction occurred and the solution was agitated overnight at 20° C. The reaction mixture was filtered; white solid was washed with chloroform.

Yield: 1.48 g (first crop)

The remaining solution was neutralized with 17.0 ml triethylamine (pH=7.5-8) and extracted with 300 ml of 3% sodium bicarbonate, the water layer was extracted with 2*30 ml of chloroform. The combined organic solution was dried with sodium sulfate and evaporated at 65° C. The product was chromatographed on a column with 300 ml of silica gel Merck Kieselgel 60 (0.040-0.063 mm), using mobile phase: EtOAc/MeOH/Et₃N 10/3/0.5 v/v, under TLC control. Desired fractions were separated, combined and evaporated.

Yield: 10.29 g (second crop)
22.76 g (third crop)

### Example 3

Preparation of Compound (2) from Compound (6a)

Reaction Scheme:

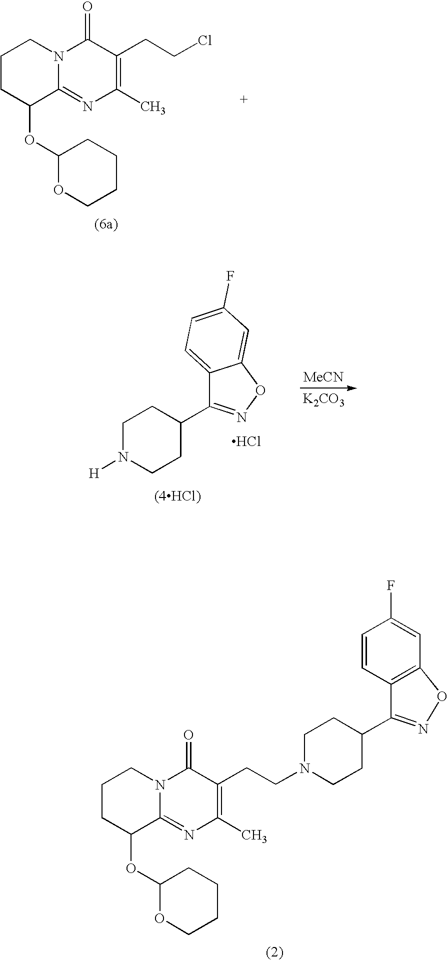

Procedure:

1.8 g of potassium iodide was suspended in 212 ml of acetonitrile and after 5 min. 21.08 g of the compound (4) hydrochloride was added. The suspension was heated to 70° C. and 35.32 g of the compound (6a) and 44.8 g of potassium carbonate was added in small portions (the reaction mixture foamed). Then the reaction mixture was heated to 80° C.

After 4 h of heating the reaction mixture was cooled to RT and agitated until the next day (15 h). Then the mixture was heated for the next 5 hours.

The suspension was filtered and washed with 2*30 ml of chloroform and the filtrate was evaporated to dryness at 80° C.

The residue (49.67 g) was dissolved in 50 ml of chloroform and the solution was extracted with 3*50 ml of 3% sodium bicarbonate, 100 ml of 10% solution of ammonium acetate and 100 ml of water. Extraction with solution of ammonium acetate and water was repeated. The organic solution was dried with sodium sulfate and evaporated at 80° C.

Yield: 43.73 g of crude (2)

### Example 4

Paliperidone Hydrochloride from Compound (2)

The crude product from the preceding example was dissolved in 100 ml of methanol and the solution was acidified with 7.5 ml of 36% HCl. The solution was agitated for 3 h at room temperature and then evaporated. The residue was dissolved in 150 ml of water and the solution was extracted with 3*40 ml of ethyl acetate. Water solution was alkalized with 50% NaOH to pH=9 and the solution was extracted with 2*80 ml of ethyl acetate, the organic solution was extracted with 3% NaHCO₃. The organic solution was after drying evaporated to dryness.

Yield: 17.60 g of crude paliperidone base.

The crude paliperidone base was dissolved in 50 ml of methanol at 60° C. and the solution was let stand to crystallize. The crystallization was finished by standing at −15° C. overnight. The crystals were filtered off and washed with 2*5 ml of cooled methanol.

Yield: 5.70 g

The mother liquor was evaporated and a second crop of paliperidone was isolated by column chromatography.

Sorbent: 1,200 g silica gel 60 (0.040-0.063 mm) Merck.

Mobile phase: EtOAc/MeOH/Et₃N 10/10/0.5 v/v

Yield: 3.0 g

Both crops were collected and dissolved in 30 ml of isopropanol, the solution was heated to 60° C. and filtered. The filtrate was acidified with 3.9M HCl in i-PrOH to pH=3 (wet pH paper). The product crystallized at 5° C. overnight in the fridge.

Yield: 5.68 g of paliperidone hydrochloride

### Example 5

Conversion of the Compound (2) into Paliperidone in Acidic Fluid

A tablet of the following composition was prepared:

| Excipient/drug substance | mg | % |
|---|---|---|
| Compound (2) | 10.8 | 10.8 |
| Prosolv HD-90 | 83.2 | 83.2 |
| Low substituted hydroxypropylcellulose | 5.0 | 5.0 |
| Sodium stearyl fumarate | 1.0 | 1.0 |
| Total | 100 | 100 |

Compound (2), Prosolv HD-90 and low substituted hydroxypropylcellulose were mixed for 10 minutes at 22 rpm in a free fall mixer. The blend was forced sieved over a 250 micron sieve. The blend was then mixed for another 10 minutes in a free fall mixer at 22 rpm. The sodium stearyl fumarate was sieved over an 800 micron sieve and added to the blend. The blend was mixed for another 5 minutes at 22 rpm. Tablets were compressed on the Ek-0. Tablet mass was 100 mg, tablet hardness 30 N and the tablet diameter was 8 mm.

The dissolution was tested under the following conditions:

Determination of the dissolution assay at single point or dissolution profile was performed in Simulated Gastric Fluid (SGF) without pepsin (pH=1.2) with the paddle apparatus based on USP <711>, “Apparatus 2”. The dissolution samples were immediately neutralized to pH=7 upon sampling. The amount of paliperidone and compound (2) in the neutralized dissolution samples at the specified time point(s) was determined by HPLC at 280 nm.

Chromatographic System:

| | |
|---|---|
| Analytical column: | Symmetry Shield, RP18 (Waters), 50 × 4.6 mm, |
| | dp = 3.5 μm |
| Guard column: | Phenomenex security guard C18 |
| Mobile phase A: | 0.04 M Acetate buffer pH = 5.0 |
| Mobile phase B | Acetonitrile |
| Column temp.: | 20° C. |
| Flow: | 1.5 ml/min (70% Mobile phase A; |
| | 30% Mobile phase B) |
| Autosampler temp.: | Ambient |
| Injection volume: | 500 μl |
| Detection: | UV, 280 nm |
| Run time: | 9 minutes |

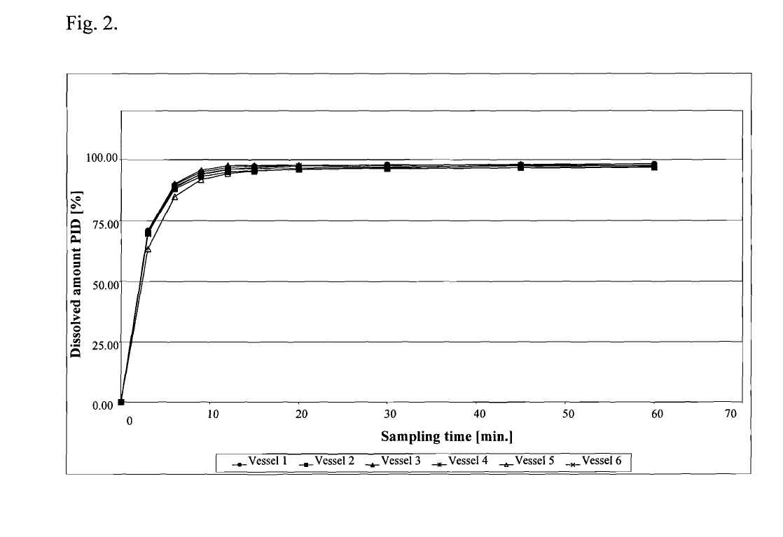

The results for a six vessel test are shown in FIGS. 1 and 2, wherein FIG. 1 shows the conversion of compound (2) into paliperidone and FIG. 2 shows the effective “release” or providing of paliperidone from the compound (2)-containing tablet.

The tablet composition is characterized by an immediate disintegration in water (similar to orally disintegratable tablets) and was chosen in order to minimize the influence of excipients in the determination of the rate of conversion in a stomach fluid.

As seen from the corresponding figures, the compound (2) quantitatively converts into paliperidone in approx. 10 minutes.

Each of the patents, patent applications, and journal articles mentioned above are incorporated herein by reference. The invention having been described it will be obvious that the same may be varied in many ways and all such modifications are contemplated as being within the scope of the invention as defined by the following claims.

1. A compound of formula (1): 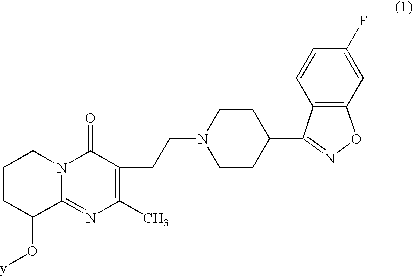
or a salt thereof, wherein Y is an acid-sensitive group of the formula: 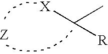
in which X represents an oxygen atom, a sulfur atom, or an —NH— group; Z represents a C2-C7 alkylene bridge; and R represents hydrogen or a C1-C4 alkyl group.

2. The compound according to claim 1, wherein X is oxygen.

3. The compound according to claim 1, wherein Z is an unbranched C3-C5 alkylene bridge.

4. The compound according to claim 1, wherein said compound of formula (1) is a compound of formula (2):

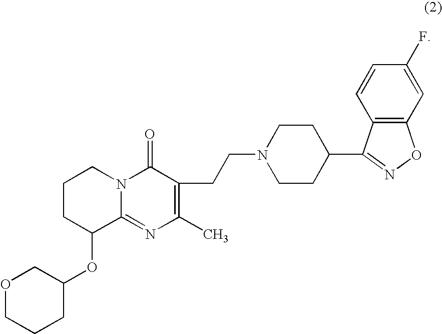

5. A pharmaceutical composition comprising a compound according to claim 1, and at least one pharmaceutically acceptable excipient.

6. A compound of formula (6) or (7), including the salts thereof: 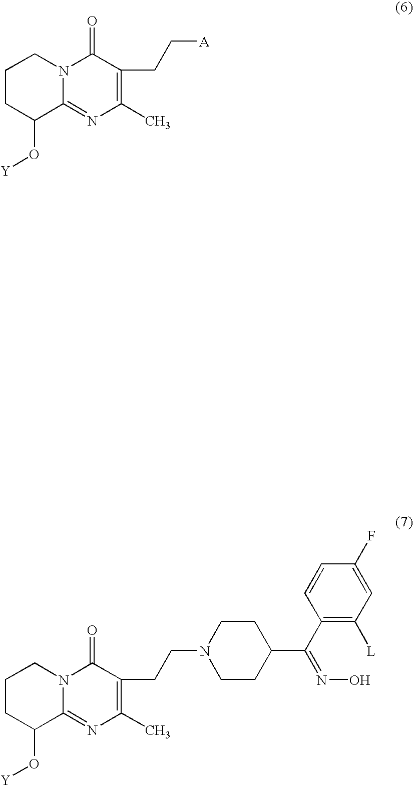
wherein Y in both formulas represents an acid-sensitive group of the formula: 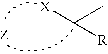
in which X represents an oxygen atom, a sulfur atom, or an —NH— group; Z represents a C2-C7 alkylene bridge; and R represents hydrogen or a C1-C4 alkyl group;
A in formula (6) represents a halogen or a sulfonyloxy group; and
L in formula (7) represents a halogen or nitro group.

7. A process which comprises reacting a Y-donor with a compound of formula (A): 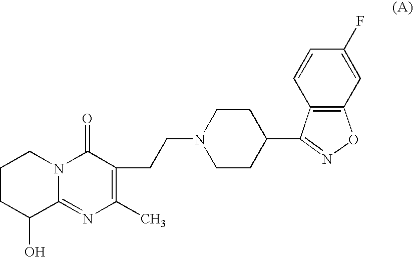
to form a compound of formula (1): 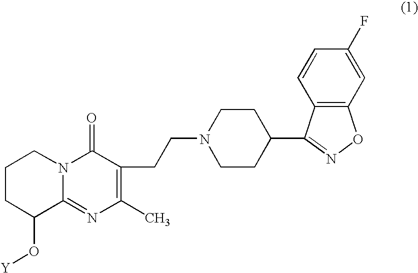
wherein Y is an acid-sensitive group of the formula: 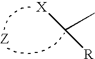
in which X represents an oxygen atom, a sulfur atom, or an —NH— group; Z represents a C2-C7 alkylene bridge; and R represents hydrogen or a C1-C4 alkyl group.

8. The process according to claim 7, wherein said Y-donor is a compound of the formula Y-LV wherein LV is a leaving group.

9. The process according to claim 8, wherein LV represents a halogen or an alkyl- or aryl-sulfonyloxy group.

10. The process according to claim 7, wherein said Y-donor is a compound of formula (Y1): 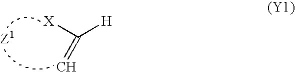
wherein Z¹ represents a C2-C6 alkylene bridge and X represents oxygen, sulfur or —NH— group.

11. The process according to claim 10, wherein said compound of formula (Y1) is a dihydropyran of formula (3): 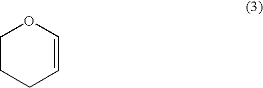
and said compound of formula (1) produced by said reaction is a compound of formula (2):

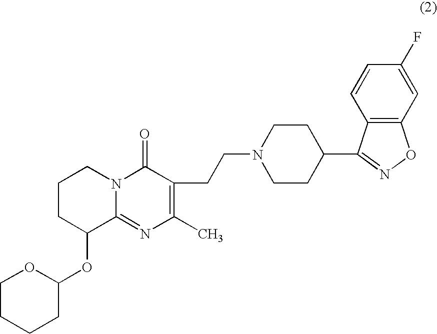

12. A process, which comprises reacting a compound of formula (6) with a compound of formula (4) to form a compound of formula (1): 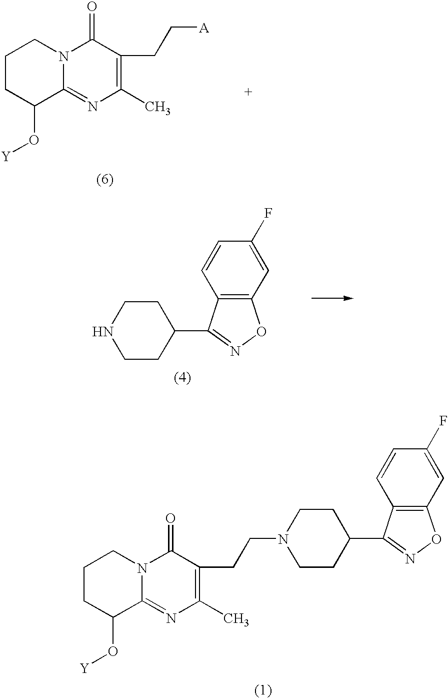
wherein A represents a leaving group and Y represents an acid-sensitive group of the formula: 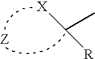
in which X represents an oxygen atom, a sulfur atom, or an —NH— group; Z represents a C2-C7 alkylene bridge; and R represents hydrogen or a C1-C4 alkyl group.

13. The process according to claim 12, wherein said A represents a halogen or a sulfonyloxy leaving group.

14. The process according to claim 13, wherein said A represents chlorine and said Y represents tetrahydropyranyl.

15. The process according to claim 12, which further comprises reacting a Y-donor with a compound of formula (5): 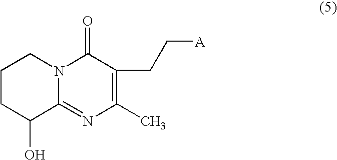
to form said compound of formula (6).

16. The process according to claim 15, wherein said A is chlorine, said Y-donor is a dihydropyran of formula (3): 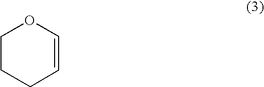
and said compound of formula (1) produced by said reaction is a compound of formula (2):

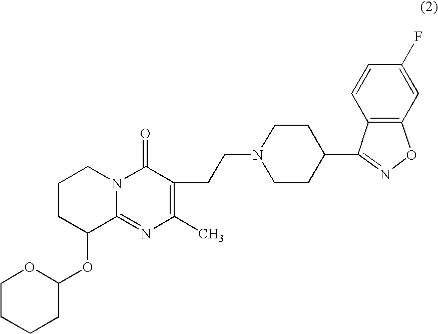

17. A process which comprises cyclizing an oxime of formula (7) to form a compound of formula (1): 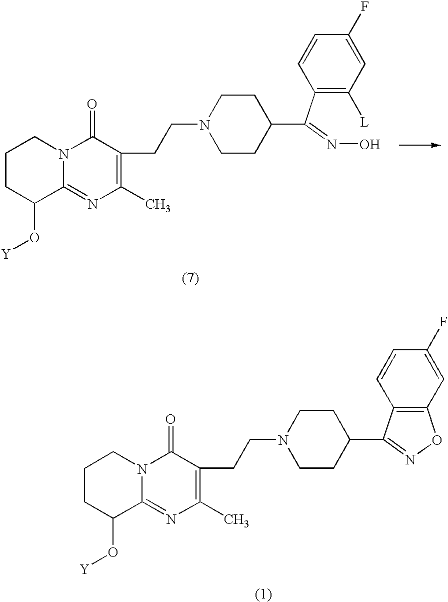
wherein Y is an acid-sensitive group of the formula: 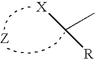
in which X represents an oxygen atom, a sulfur atom, or an —NH— group; Z represents a C2-C7 alkylene bridge; and R represents hydrogen or a C1-C4 alkyl group; and L is a reactive leaving group.

18. The process according to claim 17, wherein said L is a halogen or nitro group.

19. The process according to claim 17, which further comprises reacting a compound of formula (6) 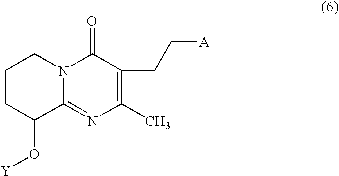
wherein A represents a leaving group;
with a compound of formula (8): 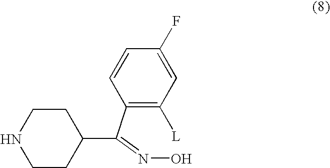
to form said compound of formula (7).

20. The process according to claim 19, wherein said A is chlorine and said L is fluorine.

21. A process for making paliperidone, which comprises subjecting a compound of formula (1) to acidic hydrolysis to form a compound of formula (A): 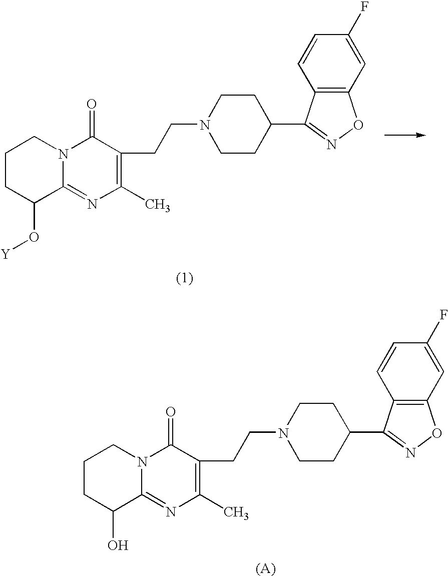
wherein Y is an acid-sensitive group of the formula: 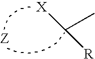
in which X represents an oxygen atom, a sulfur atom, or an —NH— group; Z represents a C2-C7 alkylene bridge; and R represents hydrogen or a C1-C4 alkyl group.

22. The process according to claim 21, wherein said Y represents a tetrahydropyranyl group and said acidic hydrolysis is carried out in the presence of hydrochloric acid.
